(19) United States
(12) Patent Application Publication
(10) Pub. No.: US 2017/0073396 A1
(43) Pub. Date: Mar. 16, 2017

(54) METHOD FOR PREPARING HUMAN PLASMA PROTEINS

(71) Applicant: LABORATOIRE FRANCAIS DU FRACTIONNEMENT ET DES BIOTECHNOLOGIES, Les Ulis (FR)

(72) Inventors: Damien BATAILLE, Ormoy (FR); Abdessatar CHTOUROU, Elancourt (FR); Patrick SANTAMBIEN, Montrouge (FR)

(21) Appl. No.: 15/125,483

(22) PCT Filed: Mar. 10, 2015
(86) PCT No.: PCT/FR2015/050598

§ 371 Date: Sep. 12, 2016

(30) Foreign Application Priority Data
Mar. 11, 2014 (FR) 1451997

(51) Int. Cl. C07K 16/06; C07K 1/22

(52) U.S. Cl. CPC C07K 16/065; C07K 1/22

(57) ABSTRACT

The invention relates to a method for preparing plasma protein concentrate from blood plasma by means of multicolumn chromatography.

The invention relates to a method for preparing human plasma proteins for therapeutic use, from blood plasma or from a plasma fraction.

### BACKGROUND OF THE INVENTION

Numerous pathologies are currently treated with blood plasma fractions enriched in one or more plasma proteins. Thus, coagulation factors are generally used in replacement therapy to prevent or treat haemorrhages associated with coagulation factor deficits. Fibrinogen is most often prescribed to treat complications associated with congenital or severe afibrinogenemia and haemorrhagic syndromes or risks of haemorrhages associated with hypofibrinogenemia. Albumin is intended to restore and maintain circulating blood volume (confirmed hypovolemia). Similarly, numerous pathologies are currently treated with blood plasma fractions enriched in immunoglobulins, in particular in immunoglobulins G (IgG), comprising generally more than 95% Ig.

For example, blood plasma fractions enriched in immunoglobulins G, or IgG concentrates, are used to correct primary immune deficiencies lacking antibody production, and certain secondary immunodeficiencies such as leukaemias, myelomas or recurrent infections, etc. Administration of Ig may also have a beneficial effect in the treatment of juvenile and adult idiopathic thrombocytopenic purpura (ITP), Guillain-Barré syndrome, demyelinating polyneuropathies, multifocal motor neuropathy, chronic inflammatory demyelinating polyradiculoneuropathies (CIDP), Kawasaki's disease, multiple sclerosis, steroid-resistant dermatomyositis, acute myasthenia, Birdshot's retinochoroiditis, neonatal jaundice due to foetal-maternal ABO incompatibility (haemolytic disease of the newborn due to ABO incompatibility), etc.

The multiple therapeutic indications, and the very high doses that may be prescribed in certain cases (up to 2 g of immunoglobulins/kg/day), have resulted in extreme stresses on supply, up to the point of shortages in Europe and in the United States of America.

Also, numerous methods have been developed for purifying plasma proteins from blood plasma. The industrial process making it possible to extract plasma proteins for therapeutic use is called “plasma fractionation.” Fractionation techniques used today generally begin with a cryoprecipitation step, consisting in thawing plasma at low temperature in order to isolate a cryoprecipitate enriched in factor VIII, von Willebrand factor, fibrinogen and fibronectin. Proteins of the supernatant fraction (cryosupernatant) may then be separated by sequential precipitations in the presence of ethanol (Cohn et al. 1946, J. Am. Chem. Soc. 68, 459).

Several variants of the original Cohn method also using ethanol fractionation have been described. Thus, the publication by Tanaka K. et al., 2000 (Braz J Med Bio Res 2000, 33(1): 27-30) describes a method for purifying immunoglobulins G from fractions “I+II+II” and “II+III” obtained after ethanol fractionation according to the Cohn method, by chromatographic separation on three types of gel, two ion-exchange gels (Q-Sepharose FF and Cm-Sepharose FF) and a gel filtration step (Sephacryl S-300 HR).

An alternative pathway to ethanol precipitation has been described by Steinbuch et al. (Rev. Franc. Et. Clin, and Biol. 1969, XIV, 1054), using precipitation with octanoic acid (or caprylic acid/caprylate). The latter precipitates most proteins from plasma and retains immunoglobulins in the supernatant. Purification of these immunoglobulins proceeds by adsorption (in a “batch”) on an anion-exchange resin, DEAE-cellulose, which also leaves immunoglobulins in the supernatant. The latter is then concentrated by ultrafiltration.

However, these ethanol or caprylic acid precipitations may cause a certain denaturation of proteins and the formation of protein aggregates (in particular immunoglobulin polymers), which may cause anaphylactic reactions. It is thus necessary to proceed to subsequent processing steps, for example with propiolactone or with reducing and alkylating agents, or at pH 4, or with PEG to precipitate aggregates. These additional steps tend to decrease the plasma protein yield.

Thus, most manufacturing methods claim an IgG yield between 3.5 and 5.5 g of IgG per litre of fractionated plasma (“A modified caprylic acid method for manufacturing immunoglobulin G from human plasma with high yield and efficient virus clearance”—Vox Sang. 2006 February; 90(2):97-104 and “A new methodology for polyvalent intravenous immunoglobulin solution production with a two-stage process of viral inactivation”—Brazilian Journal of Pharmaceutical Sciences vol. 46, n. 4, October/December, 2010).

Furthermore, the use of ethanol fractionation is relatively costly. Indeed, fractionation of a single litre of plasma requires the use of 2 l of ethanol. To process several thousand litres of plasma, it is thus necessary to store very large amounts of ethanol directly at the production site, as well as to cool the systems dedicated to ethanol fractionation. Moreover, industrial facilities must be suitable for storing, treating, eliminating and possibly recycling large volumes of solvent, which represents significant technical constraints and costs.

The purity of plasma proteins is an important issue for guaranteeing their greatest efficacy and safety during use. Thus, ethanol or caprylic acid fractionation must be supplemented by other purification steps in order to achieve the desired purity, but these operations are carried out to the detriment of the yield due to additional losses of product. For these additional steps, chromatography techniques are used more and more often because they are characterised by high degrees of purification coupled with high yields. It is thus advantageous to position these chromatography techniques as close as possible to the initial plasma in order to improve yield and purity while preserving the integrity of the proteins of interest. This is already applied for capturing relatively scarce plasma proteins such as factor XI, factor IX, factor VIII, von Willebrand factor, protein C, antithrombin III, etc. But for abundant proteins such as polyvalent immunoglobulins, albumin and fibrinogen, the volumes of chromatography gel needed to capture these molecules would be too large and would result in unprofitable industrial tools (equipment size and gel costs). Whilst some describe the accumulation of chromatography cycles (“Description and assessment of an industrial chromatography unit for preparing human plasma albumin”—Biotechnology of Blood Proteins 1993, vol 227, pp. 176-181) for processing large amounts of proteins, these practices are not optimal and remain poorly developed.

Therefore, there is still a great need to increase the production yield of plasma proteins for therapeutic use, and in particular of immunoglobulins, of albumin and/or fibrinogen, while ensuring that the final product has high purity and is free of potentially harmful contaminants.

### SUMMARY OF THE INVENTION

Generally, the present invention relates to a method for preparing a fraction of purified human plasma proteins with high yield, and in particular higher than the yield of methods known to the skilled person. To this end, the Applicant developed a method for preparing a purified human plasma protein concentrate from blood plasma, comprising at least one purification step by multicolumn chromatography, and in particular by multicolumn affinity or ion-exchange chromatography or multicolumn chromatography via hydrophobic interactions or a combination of chemical ligands (multi-modal). More precisely, the method according to the invention proposes to carry out a chromatography step by dividing the chromatography column typically used into several smaller columns, placed in series or controlled independently. Such a step implementing multicolumn chromatography makes it possible, on the one hand, to use to the maximum degree possible the functional groups present in the chromatography gel and, on the other hand, to greatly reduce the volume of chromatography gel to be used per plasma protein preparation batch.

The present invention also relates to a method for preparing a human immunoglobulin (Ig) concentrate with higher yield in comparison with methods known to the skilled person. To this end, the Applicant developed a method for preparing purified Ig, wherein the initial fractionation step using ethanol and/or caprylic acid is replaced by a capture step using multicolumn chromatography, and in particular using multicolumn affinity or ion-exchange chromatography or multicolumn chromatography via hydrophobic interactions or a combination of chemical ligands (multi-modal). No step of eliminating protein contaminants by precipitation is performed before the chromatography. Blood plasma or blood plasma cryosupernatant is directly subjected to said multicolumn chromatography. If the multicolumn chromatography is anion-exchange chromatography, the method according to the invention may advantageously comprise a fractionation step with caprylic acid and/or ethanol following said chromatography.

The invention thus relates to a method for preparing a purified plasma protein concentrate for therapeutic use from blood plasma, comprising a purification step wherein blood plasma or a plasma fraction is subjected to multicolumn chromatography.

In particular, the invention relates to a method for preparing human immunoglobulin concentrates for therapeutic use from blood plasma, comprising an immunoglobulin purification step wherein plasma or cryoprecipitated plasma supernatant is subjected to multicolumn affinity chromatography.

In another example embodiment, the immunoglobulin purification step is carried out by subjecting plasma or cryoprecipitated plasma supernatant to multicolumn anion-exchange chromatography. It is then possible to envisage a subsequent precipitation step with caprylic acid, at the conclusion of which the supernatant containing immunoglobulins is collected.

The invention also relates to a method for preparing human albumin and/or fibrinogen concentrates for therapeutic use from blood plasma, comprising an albumin and/or fibrinogen purification step wherein plasma or cryoprecipitated plasma supernatant is subjected to multicolumn chromatography, and in particular to multicolumn affinity or ion-exchange chromatography or multicolumn chromatography via hydrophobic interactions or a combination of chemical ligands (multi-modal).

According to a particular embodiment of the invention, the multicolumn affinity or ion-exchange chromatography captures the protein of interest (fibrinogen and/or albumin) which can then be eluted. According to another particular embodiment of the invention, the multicolumn affinity or ion-exchange chromatography captures the contaminants in order to collect the protein of interest (albumin and/or fibrinogen) in the non-retained fraction. According to the invention, such a method may also comprise a subsequent additional step wherein the fraction containing albumin and/or fibrinogen is subjected to a purification step by precipitation, at the conclusion of which the contaminants remain in solution.

Generally, according to the invention, it is possible to proceed to the purification step of a plasma protein for therapeutic use by multicolumn chromatography directly from blood plasma, or from cryosupernatant, or from an ethanol or caprylic acid fraction, optionally virally safe, or from any product resulting from an intermediate purification step and in particular from filtration, from another chromatography, or from multicolumn chromatography, etc.

In a particular embodiment of the invention, cryoprecipitated plasma supernatant or plasma is directly subjected to a first multicolumn chromatography in order to capture a plasma protein, then the non-retained fraction of this chromatography is subjected to a second multicolumn chromatography in order to capture a second plasma protein.

Subsequently, the non-retained fraction of the second multicolumn chromatography can be subjected to a chromatography, in particular multicolumn chromatography, or to a precipitation step in order to purify a third plasma protein.

In a particular example embodiment, the method according to the invention consists in a method for preparing an albumin and/or fibrinogen concentrate for therapeutic use from blood plasma, comprising a step of purification of cryoprecipitated plasma supernatant by multicolumn affinity or ion-exchange chromatography, optionally followed by a step of elimination of contaminants present in the fraction comprising albumin and/or fibrinogen for example by precipitation of said fraction with ethanol. In another example embodiment, the purification step by multicolumn ion-exchange chromatography or multicolumn chromatography via hydrophobic interactions or a combination of chemical ligands (multi-modal) is carried out on an immunoglobulin-depleted plasma fraction. Advantageously, the method for preparing an albumin and/or fibrinogen concentrate is carried out following a method for preparing an immunoglobulin concentrate, optionally by multicolumn chromatography, using the plasma fraction collected at the conclusion of said method.

Thus, the invention also relates to a method for fractionating blood plasma, comprising the steps consisting in directly subjecting blood plasma or cryoprecipitated plasma supernatant successively to:
multicolumn ion-exchange chromatography or multicolumn chromatography via hydrophobic interactions or a combination of chemical ligands (multi-modal) at the conclusion of which the fraction containing the immunoglobulins is collected; then
multicolumn ion-exchange chromatography or multicolumn chromatography via hydrophobic interactions or a combination of chemical ligands (multi-modal) at the conclusion of which the fraction containing albumin is collected; then
multicolumn ion-exchange chromatography or multicolumn chromatography via hydrophobic interactions or a combination of chemical ligands (multi-modal) at the conclusion of which the fraction containing fibrinogen is collected.

The sequence of the three steps can optionally be modified, in order to promote the extraction of one of the proteins with respect to the two others as required.

Thus, the invention also relates to a method for fractionating blood plasma, comprising the steps consisting in directly subjecting blood plasma or cryoprecipitated plasma supernatant successively to:
multicolumn ion-exchange chromatography or multicolumn chromatography via hydrophobic interactions or a combination of chemical ligands (multi-modal) at the conclusion of which the fraction containing the immunoglobulins is collected; then
multicolumn ion-exchange chromatography or multicolumn chromatography via hydrophobic interactions or a combination of chemical ligands (multi-modal) at the conclusion of which the fraction containing fibrinogen is collected; then
multicolumn ion-exchange chromatography or multicolumn chromatography via hydrophobic interactions or a combination of chemical ligands (multi-modal) at the conclusion of which the fraction containing albumin is collected.

The plasma fraction is thus successively subjected to three multicolumn chromatographies at the conclusion of which it is successively depleted of immunoglobulins, of albumin and then of fibrinogen, or another sequence.

In a particular example embodiment, the method according to the invention consists of a method for preparing an immunoglobulin concentrate for therapeutic use from blood plasma, successively comprising:

(a) a first viral inactivation or elimination step by physicochemical treatment of the plasma, such as a treatment with solvent/detergent or detergent alone or solvent alone without limitation of the possible combinations of the relative entities;

(b) a step of purification of cryoprecipitated plasma supernatant by multicolumn affinity chromatography of the inactivated plasma resulting from step (a);

(c) optionally a step of anti-haemagglutinin antibody elimination, in particular by multicolumn anti-A and anti-B affinity chromatography, from the immunoglobulin concentrate resulting from step (b);

(d) optionally a second viral inactivation or elimination step by nanofiltration of the immunoglobulin concentrate resulting from step (b) or (c); and

(e) a step of addition of one or more pharmaceutically acceptable stabilisers to the immunoglobulin concentrate resulting from step (b), (c) or (d).

In another particular example embodiment, the method according to the invention consists in a method for preparing an immunoglobulin concentrate for therapeutic use from blood plasma, successively comprising:

(a′) a step of purification of cryoprecipitated plasma supernatant by multicolumn affinity chromatography;

(b′) a first step of viral inactivation or elimination by detergent/solvent of the immunoglobulin concentrate obtained in step (a′);

(c′) optionally a step of anti-A and anti-B antibody elimination, in particular by multicolumn affinity chromatography, from the immunoglobulin concentrate resulting from step (b′);

(d′) optionally a second viral inactivation or elimination step by nanofiltration of the immunoglobulin concentrate resulting from step (b′) or (c′); and (e′) a step of addition of one or more pharmaceutically acceptable stabilisers to the immunoglobulin concentrate resulting from step (b′), (c′) or (d′).

In another particular example embodiment, the method according to the invention consists in a method for preparing an immunoglobulin concentrate for therapeutic use from blood plasma, successively comprising:

(A) a step of ethanol and/or caprylic acid fractionation of cryoprecipitated plasma supernatant;

(B) optionally a first step of viral inactivation or elimination by solvent/detergent of the solution obtained in step (A);

(C) a step of purification by multicolumn anion-exchange chromatography of the solution obtained in step (A) or (B);

(D) optionally a step of anti-A and anti-B antibody elimination, in particular by multicolumn affinity chromatography, from the immunoglobulin concentrate obtained at the conclusion of step (C);

(E) optionally a second viral inactivation or elimination step by nanofiltration of the immunoglobulin concentrate obtained at the conclusion of step (C) or (D); and

(F) a step of addition of one or more pharmaceutically acceptable stabilisers to the immunoglobulin concentrate resulting from step (C), (D) or (E).

The invention also relates to a method for fractionating blood plasma, comprising the steps consisting in directly subjecting blood plasma or cryoprecipitated plasma supernatant to:
multicolumn affinity chromatography wherein at least one affinity ligand is a ligand specifically binding immunoglobulins, at the conclusion of which the fraction containing the immunoglobulins is collected; and/or
multicolumn ion-exchange chromatography or multicolumn chromatography via hydrophobic interactions or a combination of chemical ligands (multi-modal), termed mixed-mode chromatography, optionally highly salt tolerant, at the conclusion of which the fraction containing fibrinogen and/or albumin is collected.

The invention also relates to a method for fractionating blood plasma, comprising the steps consisting in directly subjecting blood plasma or cryoprecipitated plasma supernatant to:
multicolumn affinity chromatography wherein at least one affinity ligand is a ligand specifically binding immunoglobulins, at the conclusion of which the fraction containing the immunoglobulins is collected; and/or
multicolumn affinity chromatography wherein at least one affinity ligand is a ligand specifically binding fibrinogen, at the conclusion of which the fraction containing fibrinogen is collected; and/or
multicolumn ion-exchange chromatography or multicolimn chromatography via hydrophobic interactions or a combination of chemical ligands (multi-modal), termed mixed mode chromatography, optionally highly salt tolerant, at the conclusion of which the fraction containing albumin is collected.

Advantageously, the multicolumn affinity chromatography targeting immunoglobulins is carried out first, the multicolumn affinity and ion-exchange chromatography or chromatographies targeting fibrinogen and/or albumin being carried out on the fraction resulting from this first fractionation step and depleted of immunoglobulins type G. The multicolumn chromatography for capture of albumin can advantageously be carried out in third position.

The invention also relates to an immunoglobulin type G concentrate having an immunoglobulin distribution profile similar or identical to the immunoglobulin distribution profile in plasma.

Preferentially, the immunoglobulin concentrate according to the invention is an IgG immunoglobulin concentrate having between 50 and 70% IgG1, 25 to 35% IgG2, 2 to 8% IgG3 and 1 to 8% IgG4.

The invention further relates to an immunoglobulin concentrate having an antigenic repertoire similar or identical to the antigenic repertoire of plasma. Preferentially, the immunoglobulin concentrate according to the invention has an antigenic repertoire similar and/or superior to the antigenic repertoire of concentrates of the prior art.

### BRIEF DESCRIPTION OF THE FIGURES

FIG. 1 (FIGS. 1A-1D) represents diagrammatically the principle of the multicolumn chromatography as implemented in the method according to the invention;

FIG. 2 represents diagrammatically the sequence of the steps for the continuous purification of abundant plasma proteins by means of a succession of multicolumn chromatographies, according to an example of implementation of the method according to the invention;

FIG. 3 is an image of an SDS-PAGE gel under non-reducing conditions and Coomassie blue staining of the fractions obtained at the conclusion of the multicolumn chromatographies carried out in Examples 2, 3 and 4.

### DETAILED DESCRIPTION

### Definitions

By “plasma protein” is meant according to the invention any protein, and more particularly any protein of industrial or therapeutic interest, contained in blood plasma. Blood plasma proteins include albumin, alpha/macroglobulin, antichymotrypsin, antithrombin, antitrypsin, Apo A, Apo B, Apo C, Apo D, Apo E, Apo F, Apo G, beta XIIa, C1-inhibitor, C-reactive protein, C7, C1r, C1s, C2 C3, C4, C4bP, C5, C6, C1q, C8, C9, carboxypeptidase N, ceruloplasmin, factor B, factor D, factor H, factor I, factor IX, factor V, factor VII, factor VIIa, factor VIII, factor X, factor XI, factor XII, factor XIII, fibrinogen, fibronectin, haptoglobin, haemopexin, heparin cofactor II, histidine rich GP, IgA, IgD, IgE, IgG, ITI, IgM, kininase II, kininogen HPM, lysozyme, PAI 2, PAI I, PCI, plasmin, plasmin inhibitor, plasminogen, prealbumin, prekallikrein, properdin, protease nexin INH, protein C, protein S, protein Z, prothrombin, serum amyloid protein (SAP), TFPI, thiol-proteinase, thrombomodulin, tissue factor (TF), TPA, transcobalamin II, transcortin, transferrin, vitronectin, and von Willebrand factor.

In particular, plasma proteins include coagulation proteins, i.e., plasma proteins involved in the chain reaction cascade leading to the formation of a blood clot. Coagulation proteins include factor I (fibrinogen), factor II (prothrombin), factor V (proaccelerin), factor VII (proconvertin), factor VIII (anti-haemophilic factor A), factor IX (anti-haemophilic factor B), factor X (Stuart factor), factor XI (Rosenthal factor or PTA), factor XII (Hageman factor), factor XIII (fibrin stabilising factor or FSF), PK (prekallikrein), HMWK (high-molecular-weight kininogen), factor III (thromboplastin or tissue factor), heparin cofactor II (HCII), protein C (PC), thrombomodulin (TM), protein S (PS), von Willebrand factor (WF) and tissue factor pathway inhibitor (TFPI), or tissue factors.

In certain embodiments, the plasma protein consists of a coagulation protein with enzymatic activity. Coagulation proteins with enzymatic activity include activated forms of factor II (prothrombin), factor VII (proconvertin), factor IX (anti-haemophilic factor B), factor X (Stuart factor), factor XI (Rosenthal factor or PTA), factor XII (Hageman factor), factor XIII (fibrin stabilising factor or FSF) and PK (prekallikrein).

In the context of the invention, the term “human immunoglobulins” or “human Ig” refers to polyvalent immunoglobulins that may be immunoglobulins A (IgA), immunoglobulins E (IgE), immunoglobulins M (IgM) or immunoglobulins G (IgG). The human immunoglobulins according to the invention are advantageously IgG, regardless of subclass (IgG1, IgG2, IgG3 and IgG4). They may be whole immunoglobulins, or any intermediate fraction obtained during the polyvalent immunoglobulin manufacturing process.

By “plasma fraction” is meant any part or sub-part of plasma, having been subjected to one or more purification steps. Plasma fractions thus include cryoprecipitated plasma supernatant, plasma cryoprecipitate (resuspended), fractions I to V obtained by ethanol fractionation (according to the Cohn or Kistler & Nitschmann method), supernatant and precipitate obtained after precipitation with caprylic acid and/or caprylate, eluates of chromatographies and unadsorbed fractions of chromatography columns, including multicolumn chromatography and filtrates.

According to the invention, “cryoprecipitated plasma supernatant” or “cryosupernatant” corresponds to the liquid phase obtained after thawing frozen plasma (cryoprecipitation). In particular, the cryosupernatant may be obtained by freezing blood plasma at a temperature between −10° C. and −40° C., then gentle thawing at a temperature between 0° C. and +6° C., preferentially between 0° C. and +1° C., followed by centrifugation of the thawed plasma in order to separate the cryoprecipitate and the cryosupernatant. The cryoprecipitate is concentrated in fibrinogen, fibronectin, von Willebrand factor and factor VIII, whereas the cryosupernatant contains complement factors, vitamin K dependent factors such as protein C, protein S, protein Z, factor II, factor VII, factor IX and factor X, fibrinogen and immunoglobulins and albumin.

By “purification step” is meant any step of a method making it possible to enrich a product of interest, and in particular a plasma protein, in a given fraction.

The preparation method according to the invention rests principally on the use of a multicolumn chromatography step for purifying a plasma protein for therapeutic use from blood plasma or from any plasma fraction. The implementation of such a multicolumn chromatography step is particularly suited for the preparation of an immunoglobulin concentrate. Indeed, it is possible, according to the invention, to directly subject human blood plasma or human blood plasma cryosupernatant to multicolumn chromatography. Of course, it is also possible to envisage a step of filtration and/or ethanol/caprylic acid fractionation upstream of the purification by multicolumn chromatography.

Use of such multicolumn chromatography generally allows a reduction on an industrial scale of the cost of plasma proteins for therapeutic use. In particular, multicolumn chromatography makes it possible to use the chromatographic support at total saturation of said columns, which requires a smaller amount of gel than conventional chromatography with which customary practice is to limit the capacity as soon as 10% leakage of molecule of interest is detected. Similarly, since the elution, washing and sanitisation phases are carried out on smaller columns, solution and buffer needs are greatly reduced in comparison with conventional chromatography. Whilst the principle of fractionation of plasma molecules by a cascade of chromatographies has already been described (John Curling et al. “A comparative study of Cohn and chromatographic fractionation using a novel affinity “Cascade Process”” PPB congress May 2005), it is of only limited industrial interest because of associated low capacities and productivities. In a particularly surprising and advantageous manner, use of the multicolumn chromatography according to the invention makes the principle of cascade chromatography much more attractive, by allowing greater capture capacity and/or higher productivity expressed in unit of mass of product extracted per unit of time and volume of gel.

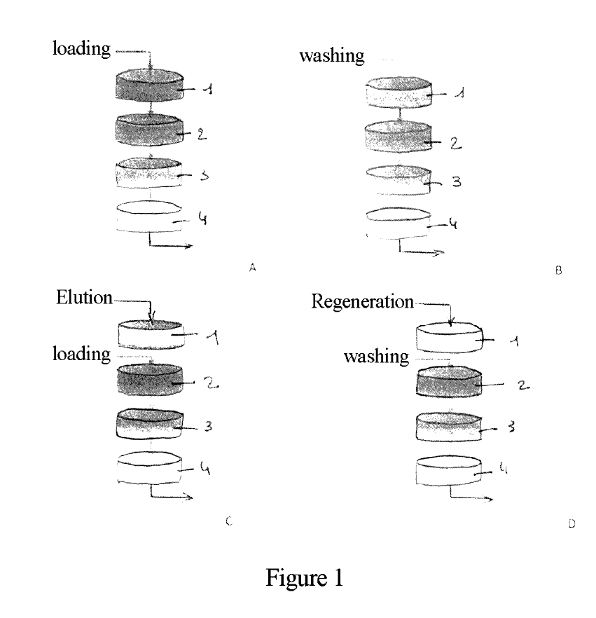

The principle of multicolumn chromatography is represented diagrammatically in FIG. 1. Such multicolumn chromatography allows the fractionation of a column (a chromatography support) conventionally used in chromatography, into several columns (of the same chromatography support) which are of smaller size and are linked to one another so that the outlet of one is connected to the inlet of the other.

Generally, multicolumn chromatography excludes the juxtaposition (in series or in parallel) of several chromatography columns of different natures.

In summary, the fraction to be purified is injected onto a column 1 (FIG. 1A), the outlet of which is connected in series to a column 2. Thus, the leakages of the plasma protein of interest leaving column 1, instead of going into a “non-adsorbed” collected fraction are collected on column 2 (guard column) onto which the residual plasma protein of interest will be able to adsorb. The number of guard columns 2, 3, 4, etc., placed in series following column 1 depends on the proportion of leakage of protein of interest to be collected (in the example described here, 3 guard columns 2-4 are represented). Once the loading and then the washing of column 1 has been completed (FIGS. 1A and 1B), it can be separated from the other columns 2-4, in order to individually undergo elution (FIG. 1C), regeneration (FIG. 1D) and optionally equilibration (not represented) steps. Once column 1 has been reequilibrated, it can be placed in series again, as final guard column, etc.

In parallel, column 2, which is partially loaded, then moves into first position and in turn undergoes loading of plasma fraction, the leakages of which are again collected on the following guard columns 3 and 4, and so on.

The multicolumn chromatography thus makes it possible to optimize the saturation of the gel without losing material by breaking through, since the leakages are collected on the guard columns.

According to the invention, several multicolumn chromatography techniques may be used. Known in particular is Stimulated Moving Bed (SMB) technology, from which is derived Sequential Multicolumn Chromatography (SMCC) technology, which are particularly suitable for implementation of the method according to the invention. Examples of embodiments of multicolumn chromatographies are described in patent applications WO2007/144476 and WO2009/122281.

According to the invention, the multicolumn chromatography may be affinity, ion-exchange (anions or cations), hydrophobic interaction, mixed-mode or size-exclusion chromatography. Preferentially, the multicolumn chromatography is multicolumn affinity, mixed-mode or anion-exchange chromatography.

In a particular example embodiment of the invention, the multicolumn chromatography is radial chromatography, i.e., using radial columns. According to the invention, it is possible to use radial columns having a ratio of at least two between the surface area of the larger outer diameter (inlet side) and the surface area of the smaller inner diameter (outlet side). These columns make it possible to increase load flow rates, making the method particularly advantageous when there are large volumes of plasma to be processed and/or when the plasma protein of interest, such as immunoglobulins, must be purified quickly in order to preserve its molecular integrity. Similarly, this makes it possible to reduce the duration of the purification steps and thus to multiply the number of batches per unit of time.

Depending on needs, and more particularly on the volumes of plasma to be processed, chromatography columns of a few millilitres (for implementation on a laboratory scale, for example), for example from 5 to 20 ml, to several hundreds or thousands of litres (on an industrial scale), for example from 200 to 20001, can be used. For each range of columns, it is also possible to adapt the height of the gel bed forming the stationary phase, for example 6 cm, 12 cm or 18 cm. The skilled person knows how, depending on the volumes to be processed and/or the desired flow rate, to adapt the number of columns, their dimensions and the heights of the gel.

In a particular example, and in particular for preparing an immunoglobulin concentrate directly from cryosupernatant, multicolumn affinity chromatography is advantageously used.

It is known that most affinity gels (forming the stationary phase of affinity chromatography), in particular those used industrially, have a maximum immunoglobulin (Ig) loading capacity four to five times smaller than ion-exchange gel (20-30 g/l versus 80-100 g/l). It is thus generally acknowledged that to process large volumes of plasma the quantity of affinity gel required makes the process economically unviable.

However, the Applicant discovered that this disadvantage may be countered by dividing the chromatography column into several smaller columns, in particular into 3 to 8 columns, preferentially 3 to 5 columns. The use of multiple chromatography columns makes it possible to place in series of one or more principal column(s), guard columns which will capture the plasma proteins of interest that escaped the principal column(s) during the loading of the protein of interest. The affinity ligands present in this (these) principal column(s) are then saturated so as to reach the maximum capacity of the chromatography gel. This (these) column(s) is (are) then taken out of the system to be eluted separately and to allow the proteins of interest to be collected. The guard columns then in turn become the principal column(s). From 3 to 50 successive cycles, preferably from 5 to 30 and more preferably from 7 to 15, make it possible to maximise the use of affinity ligands and thus to reduce the total volume of gel needed to capture and elute the molecule of interest.

Advantageously, the affinity ligand is selected from antibodies, antibody fragments, antibody derivatives or chemical ligands such as peptides, mimetic peptides, peptoids, nanofitins or oligonucleotide ligands such as aptamers.

For example, a gel comprising a crosslinked agarose matrix, making it possible to work with high flow rates, may be used in combination with an affinity ligand able to bind the protein of interest. In a particular embodiment, the affinity ligand makes it possible to bind the Fc fragment of human Igs or any conserved sequence within immunoglobulins.

In another particular embodiment, the affinity ligand used is a recombinant protein recognising the Fc fragment of Igs and coupled to a matrix, for example IgSelect gel from GE Healthcare.

Advantageously, the ligand may be selected so as to specifically recognise a class of immunoglobulins (for example immunoglobulins G) and/or to specifically recognise one or more immunoglobulin subclasses (for example, IgG1 and/or IgG2 and/or IgG3 and/or IgG4). In another particular embodiment, the affinity ligand used is protein G, which has affinity for IgGs and does not advantageously have affinity for IgAs. In still another particular embodiment, the affinity ligand used is protein A, which specifically binds IgG1, IgG2 and IgG4 but does not allow capture of IgG3.

In a particular example embodiment, the affinity ligand of the multicolumn affinity chromatography has affinity for immunoglobulins G, and is advantageously selected from ligands having affinity for IgG1, IgG2, IgG3 and IgG4. More particularly, the IgG concentrate obtained according to the method of the invention advantageously has an IgG subclass distribution profile similar to that of plasma. In particular, the IgG concentrate according to the invention advantageously comprises between 50 and 70% IgG1, 25 to 35% IgG2, 2 to 8% IgG3 and 1 to 8% IgG4, or more preferably between 60 and 70% IgG1, 30 to 35% IgG2, 3 to 6% IgG3 and 2 to 5% IgG4. In a particular embodiment, the concentrate obtained according to the method of the invention may have a small decrease in IgG3 and/or IgG4 subclasses in comparison with plasma, the product remaining nevertheless comparable in therapeutic efficacy to a product having an IgG subclass repertoire similar to that of plasma.

In another particular example embodiment, the affinity ligand of the multicolumn affinity chromatography has affinity for fibrinogen or albumin. More particularly, the invention also relates to a method for preparing human fibrinogen and/or albumin concentrates directly from blood plasma, wherein said plasma or cryoprecipitated plasma supernatant is subjected to multicolumn affinity chromatography wherein the affinity ligand(s) has (have) affinity for fibrinogen and/or albumin. The ligand used may be in particular any commercially available ligand, for example the CaptureSelect HSA (Life Technologies) affinity ligand or the CaptureSelect Fibrinogen (Life Technologies) affinity matrix ligand.

Advantageously, the affinity ligand selected for the method of the invention is resistant to the conditions of sanitisation and/or intensive re-use compatible with industrial use. In a particular embodiment, the affinity ligand is thus advantageously selected from peptides and/or peptoids (resulting from combinatorial biology, such as phage display), nanofitins (extracted from extremophile bacteria), and/or aptamers.

In a particular example, the affinity ligand is an aptamer, and in particular a nucleic aptamer. The term “aptamer” as used herein refers to a single-stranded nucleic acid molecule, DNA or RNA, and in particular a single-stranded nucleic acid molecule able to specifically bind to the protein of interest, for example to an immunoglobulin by binding to the Fc fragment of human Igs or to the conserved sequences of the immunoglobulin. Advantageously, the aptamer may be selected so as to specifically recognise a class of immunoglobulin (immunoglobulins G, for example) and/or to specifically recognise one or more immunoglobulin subclasses (for example, IgG1 and/or IgG2 and/or IgG3 and/or IgG4). Aptamers generally comprise between 5 and 120 nucleotides and may be selected in vitro by the so-called SELEX (Systematic Evolution of Ligands by Exponential Enrichment) method. By “nucleic aptamer” is meant according to the invention a single-stranded nucleic acid, and in particular a single-stranded nucleic acid specifically binding to the Fc fragment of human IgGs.

Aptamers have numerous advantages. Due to their oligonucleotide nature, aptamers have low immunogenicity and high resistance to stringent physicochemical conditions (presence of DMSO, very acidic or very basic pH, use of organic solvents or high temperature) allowing varied sanitisation strategies in the context of use as an affinity ligand. It is thus possible to increase the lifespan of affinity gels by proceeding to sanitisation steps in order to clean the chromatography columns and to limit their fouling and to reduce the risks of viral or prion contamination, despite the multiple cycles and higher loads in comparison with conventional chromatography. Aptamers, by virtue of their nucleic acid type structure, are particularly suitable for sanitisations at basic pH, allowing re-uses of 100 to 200 cycles.

Patent application FR 2 970 003, in the name of the Applicant, describes methods for manufacturing affinity supports with immobilised nucleic aptamers. In particular, this application describes a method for immobilising nucleic acids comprising at least one reactive amine functional group, by grafting on a solid support having at its surface activated carboxylic acid groups.

In a particular embodiment, the multicolumn chromatography may advantageously be applied to one or more successive purification steps, using successive chromatography columns. Thus, the fraction not retained by the first multicolumn chromatography may be used to purify other plasma proteins of interest in the most suitable order. In a particular example embodiment, a first multicolumn chromatography is carried out on blood plasma or cryoprecipitate in order to purify the immunoglobulins. The plasma fraction resulting from this first purification is subjected to a second multicolumn chromatography in order to purify albumin. The plasma fraction resulting from this second multicolumn chromatography, depleted of immunoglobulins and of albumin, can then be subjected to a third multicolumn chromatography in order to purify fibrinogen, etc. It is in particular possible to treat the plasma in continuous flow, so as to continuously purify the immunoglobulin, albumin and/or fibrinogen in particular, without activating the coagulation cascade.

In another particular example embodiment, a first multicolumn chromatography is carried out on blood plasma or cryoprecipitate in order to purify the immunoglobulins. The plasma fraction resulting from this first purification is subjected to a second multicolumn chromatography in order to purify fibrinogen. The plasma fraction resulting from this second multicolumn chromatography, depleted of immunoglobulins and of fibrinogen, can then be subjected to a third multicolumn chromatography in order to purify albumin, etc. It is in particular possible to treat the plasma in continuous flow, so as to continuously purify the immunoglobulin, fibrinogen and/or albumin in particular, without activating the coagulation cascade.

According to the invention, the method for preparing human plasma protein concentrates for therapeutic use may also comprise at least one of the following steps:

(i) a viral inactivation or viral elimination step;

(ii) an ion-exchange chromatography step;

(iii) an anti-A and anti-B antibody elimination step, in particular by affinity chromatography;

(iv) a precipitation step with caprylic acid and/or caprylate;

(v) a concentration step by ultrafiltration;

(vi) a formulation step.

Advantageously, the preparation method according to the invention makes it possible to obtain a human protein concentrate for therapeutic use and may comprise at least one subsequent step among steps (i) to (vi).

In a particular example embodiment, the preparation method makes it possible to obtain a human immunoglobulin concentrate for therapeutic use, optionally comprising one or more subsequent steps among steps (i) to (vi).

In another particular example embodiment, the preparation method makes it possible to obtain an albumin concentrate for therapeutic use, said method optionally comprising one or more subsequent steps among steps (i) to (vi).

In another particular example embodiment, the preparation method makes it possible to obtain a fibrinogen concentrate for therapeutic use, said method optionally comprising one or more subsequent steps among steps (i) to (vi).

For example, the human plasma protein concentrate and/or immunoglobulin concentrate obtained by directly subjecting blood plasma or cryosupernatant to multicolumn chromatography, in particular multicolumn affinity or anion-exchange chromatography, may advantageously undergo at least one step of elimination or inactivation of at least one infectious agent.

Among the infectious agents targeted in step (i), mention may be made of viruses and UTAs (unconventional transmissible agents) such as prions.

Viral inactivation often comprises treatment with chemicals, for example with solvent and/or detergent, and/or with heat (pasteurisation and/or dry heating) and/or by irradiation (gamma and/or UV-C) and/or by pH treatment (treatment at acidic pH).

Preferably, step (i) according to the invention comprises at least one treatment with solvent and detergent. Treatment with solvent and detergent (generally called solvent/detergent or S/D treatment) comprises in particular treatment with tri-n-butylphosphate (TnBP) and/or detergent selected from Triton X-100, Tween (preferably Tween 80), sodium cholate and 2-[4-(2,4,4-trimethylpentane-2-yl)phenoxy]ethanol (Octoxinol).

Nanofiltration may also be used to remove infectious agents, in particular viruses and UTAs. In the case of plasma proteins, nanofiltration generally refers to filtration of the protein concentrate of interest through a filter of pore size smaller than 80 nm. For example, the following filters are available: BioEx, Planova® 75 nm, Planova® 35 nm, Planova® 20 nm or Planova® 15 nm (Asahi corporation), Ultipor DV 50 or DV 20 (Pall Corporation), Virosart CPV (Sartorius), Viresolve NFR or NFP (Millipore). Nanofiltration may advantageously be carried out on a single filter or on several filters in series of identical or decreasing pore size.

Infectious agents may also be removed by means of depth filtration. Available filters are, for example, filters composed of regenerated cellulose, wherein filtration aids may be added (such as Celite, pearlite or Kieselguhr) marketed by Cuno (Zeta+VR series filters), Pall-Seitz (P-series Depth Filter) or Sartorius (Virosart CPV, Sartoclear P depth filters).

In a particular embodiment, a viral inactivation step may advantageously be carried out directly on plasma or cryoprecipitated plasma supernatant or resuspended plasma cryoprecipitate, so that the totality of proteins to be purified benefit from treatment upstream of the multicolumn chromatography.

In another particular embodiment, crude plasma and/or cryoprecipitated plasma supernatant and/or resuspended plasma cryoprecipitate may be subjected to depth filtration or to a filtration sequence, for example on a polypropylene filter or equivalent media of 6 μm, 1 μm, then 0.45-0.2 μm, upstream of the multicolumn chromatography. Such a preliminary step advantageously makes it possible to prevent and/or reduce premature fouling of columns used for multicolumn chromatography. In another particular embodiment, the depth filter(s) used is a (are) delipidation depth filter(s) (Cuno or Pall-Seitz, for example) allowing a reduction of lipids in the processed plasma fraction.

Advantageously, viral inactivation or elimination step (i) is followed by ion-exchange chromatography step (ii).

In a particular example, ion-exchange chromatography step (ii) is anion- or cation-exchange chromatography. Such anion- or cation-exchange chromatography steps are described in patent applications EP 0 703 922 and WO 99/64462 in the name of the Applicant.

In a particular example, anion-exchange chromatography step (ii) may be implemented on crosslinked polysaccharide gel or vinyl or acrylic polymer gel, grafted with DEAE or TMAE or QAE groups, as described in patent applications WO2002/092632 and WO2013/007740 in the name of the Applicant.

In another particular example, multicolumn anion-exchange and/or mixed-mode chromatography is carried out on a highly salt tolerant matrix making it possible to capture the plasma protein in a medium having high salinity such as the plasma or fraction not retained by a chromatography. Advantageously, highly salt tolerant multicolumn anion-exchange and/or mixed mode chromatography is carried out on STAr AX gel from PALL BIOSEPRA, Capto MMC gel from GE Healthcare or ESHMUNO HCX gel from Merck, or any equivalent gel known to the skilled person.

In particular, for the preparation of an immunoglobulin concentrate for therapeutic use, anion-exchange chromatography step (ii) may comprise:
adjusting to pH 8 to 10 the solution having undergone solvent-detergent treatment (step (i));
loading thereof onto the chromatography column equilibrated beforehand in buffer to pH 8 to 10, which allows adsorption of immunoglobulins and passage of unadsorbed proteins in the effluent;
washing with the same buffer until all unadsorbed proteins and the solvent/detergent mixture are removed;
and eluting immunoglobulins with a suitable buffer.

This elution may be carried out with phosphate buffer at pH between 4 and 7, and preferably at pH 6.2 to elute immunoglobulins.

The method according to the invention may also comprise an anti-A and/or anti-B antibody elimination step (iii). In a particular example, anti-A and/or anti-B antibody elimination step (iii) is carried out on the solution obtained at the conclusion of step (ii). This step may, for example, be carried out according to the method described in patent application WO 2007/077365 in the name of the Applicant. Thus, in the case of the preparation of an immunoglobulin concentrate for therapeutic use, the solution obtained in step (ii) may be subjected to an anti-A and anti-B antibody elimination step by immunoaffinity chromatography by percolation of said polyvalent immunoglobulin concentrate on a support whose matrix is grafted with oligosaccharide groups antigenically similar to blood groups A and/or B, or on a mixture of supports whose matrices are grafted with oligosaccharide groups antigenically similar to blood groups A and/or B. In a particular embodiment, anti-A and/or anti-B antibody elimination step (iii) is carried by multicolumn affinity chromatography.

In certain cases, the method according to the invention may comprise a precipitation step (iv) with caprylic acid and/or caprylate. Generally, adding caprylic acid and/or caprylate, in slightly acidic medium, makes it possible to precipitate plasma proteins, except for Igs which remain in the supernatant. According to the invention, it is possible to proceed to this caprylic acid precipitation step (iv) before or after the purification step by multicolumn chromatography.

In a particular example embodiment, the purification step of the plasma protein of interest by multicolumn anion-exchange chromatography is followed by a subsequent caprylic acid and/or caprylate precipitation step. Such a combination of multi column ion-exchange chromatography/caprylic acid precipitation may be particularly advantageous for preparing an Ig concentrate (particularly an IgG concentrate) and/or an albumin and/or fibrinogen concentrate.

In particular, for preparing an immunoglobulin concentrate for therapeutic use, precipitation step (iv) with caprylic acid may comprise steps consisting in:
optionally adjusting the pH of the plasma fraction to a value between 3.0 and 6.0, preferentially to pH 4;
adding caprylic acid to the plasma fraction;
centrifuging or filtering to collect the IgG-enriched supernatant.

The method according to the invention may advantageously comprise one or more concentration steps (v) by ultrafiltration. For example, during the preparation of an Ig concentrate, it is possible to subject the Ig-enriched fraction, resulting from the multicolumn chromatography step, optionally subjected beforehand to one and/or other of steps (i) to (iv), to membrane ultrafiltration.

It is also possible to envisage step (vi) of adding one or more pharmaceutically acceptable stabilisers to the human plasma protein concentrate. According to the invention, it is possible to envisage an additional viral security step (i), by nanofiltration, before formulation step (vi).

By pharmaceutically acceptable stabilisers is meant formulations suitable for plasma protein concentrates, in particular excipients as described in applications FR 03 08403, and preferentially formulations suitable for immunoglobulin concentrates, and in particular excipients as described in applications FR 03 04388, FR 08 59117, FR 10 54721 and FR 10 55825 in the name of the Applicant.

Formulation step (vi) may optionally be followed by a step (vii) of freezing or freeze-drying of said pharmaceutical preparation obtained in step (vi).

Advantageously, the method according to the invention allows the preparation of immunoglobulin concentrates, and/or fibrinogen and/or albumin concentrates, in particular by avoiding cold storage steps.

Advantageously, the method according to the invention makes it possible to obtain an immunoglobulin concentrate with a yield superior to 5 g/l of plasma, preferably with a yield superior to 6 g/l of plasma. In a very advantageous manner, the method according to the invention is optimised in order to obtain an immunoglobulin yield close to 7-8 g/l of initial plasma.

Advantageously, the method according to the invention makes it possible to obtain an albumin concentrate with a yield superior to 30 g/l, very advantageously superior to 35 g/l. With regard to fibrinogen, since the plasma level is about 3 g/l, an affinity capture step advantageously makes it possible to capture 80% thereof in one step. Preferably, the continuous chromatography makes it possible to achieve yields superior to 90%.

In an advantageous manner, the immunoglobulin concentrate obtained by the method according to the invention has an antigenic repertoire similar or identical to the antigenic repertoire of plasma. In a particular embodiment, the immunoglobulin concentrate obtained by the method according to the invention has an antigenic repertoire similar and/or superior to the antigenic repertoire of concentrates of the prior art. Indeed, the limited losses of immunoglobulins during the method according to the invention advantageously make it possible to retain an immunoglobulin distribution similar to that of plasma and to preserve a broad antigenic panel.

The method according to the invention is thus particularly advantageous for producing fractions of immunoglobulins directed against a specific antigenic target.

Likewise, the method according to the invention is particularly advantageous for the preparation of an albumin concentrate, in particular from a plasma fraction already depleted of immunoglobulins. The step of multicolumn chromatography according to the invention enables a capture yield of more 90%. Although the capacity of the gel during a conventional chromatography step is generally between 25 and 30 g/l of gel, the use of multicolumn anion-exchange or hydrophobic-interaction chromatography or a combination of chemical ligands (multi-modal) according to the invention makes it possible to obtain a capacity of more 50 g/l of gel, i.e. total saturation of the dynamic capacity of the support.

The following examples illustrate the invention, without however limiting its scope.

### Example 1

### Preparation of Immunoglobulin Concentrates by Multicolumn Affinity Chromatography on Cryosupernatant

Materials & Methods

Cryosupernatant

Two batches of about 5 litres of human plasma cryosupernatant (batches 13500-13L-06002 and 13500-13L-06007) comprising between 8 and 10 g/l of IgG are aliquoted into 700 ml fractions then frozen at −80° C. The characteristics of the batches used are presented in Table 1 below.

TABLE 1
Distribution of IgG subclasses in the cryosupernatants

| | | Total IgGs | IgG1 | IgG2 | IgG3 | IgG4 |
|---|---|---|---|---|---|---|
| Raw material | Batch no. | (%) | (%) | (%) | (%) | (%) |
| Cryosupernatant | 13L06002 | 100.0 | 57.9 | 32.0 | 3.5 | 6.6 |
| Cryosupernatant | 13L06007 | 100.0 | 57.2 | 32.3 | 4.0 | 6.6 |

During a test, the required volume of cryosupernatant is thawed for 20-30 minutes in a 37° C. water bath with the product not exceeding an internal temperature of 25° C.

Buffer Solutions

The compositions of the buffer solutions used during the various steps of the affinity chromatography method are summarised in Table 2 below.

TABLE 2
Buffer solutions for affinity chromatography

| Phases | Composition | Target values |
|---|---|---|
| Equilibration and | Na₂HPO₄•12H₂O: 16.7 mM | pH = 7.4 |
| washing | NaH₂PO₄•2H₂O: 3.34 mM | |
| | 150 mM NaCl | |
| Elution | 0.1M Glycine | pH = 3 |
| Eluate adjustment | 0.1M Glycine | pH = 11 |

The compositions of the buffer solutions used during the various steps of the ion-exchange chromatography method are summarised in Table 3 below.

TABLE 3
Buffer solutions for ion-exchange chromatography.

| Phases | Composition | Target values |
|---|---|---|
| Pre-equilibration | Glycine•NaCl | pH: 9.0 ± 0.1 |
| | | conductivity: 8.5 ± 0.5 mS/cm |
| Equilibration | Glycine•NaCl | pH: 9.0 ± 0.1 |
| and washing | | conductivity: 1000 ± 100 μS/cm |
| Elution | Na₂HPO₄•12H₂O: | pH: 6.2 ± 0.1 |
| | NaH₂PO₄•2H₂O | conductivity: 1600 ± 100 μS/cm |

Chromatography Gels

For affinity chromatography, an IgSelect® gel from GE Healthcare is used (batch nos. 10035817 and 10017479). The affinity ligand used is a ligand from BAC (BioAffinityCompany), which specifically binds the Fc fragment of human IgGs. This ligand is a 14 kD recombinant protein, coupled with the matrix base via a long carbon spacer that facilitates the adsorption of Igs. The latter is coupled with the spacer via multipoint amide bonds.

For ion-exchange chromatography, a strong anion-exchange gel, Fractogel® EMD TMAE, is used (batch no. 09L03583). This gel consists of crosslinked polymethacrylate resin on which are grafted trimethylaminoethyl (TMAE) groups.

Columns

For multicolumn affinity chromatography, 3 to 5 radial columns from the company Proxcys, references μ-RFC 5.0-6.0 (batch 1303B014—gel volume: 10 ml; gel height: 12 cm; ratio of inlet diameter to outlet diameter: 2:1) were used in combination with a BioSc Lab automated system (Novasep).

For TMAE chromatography, an XK 16 column (GE Healthcare) with gel volume 41 ml, height 20.4 cm, and surface area 2.0 cm² was used with an AKTA Purifier 10 automated system (GE Healthcare).

Ultrafiltration

A Millipore Biomax 30 (PES) cassette with a 30 kDa cut-off and a surface area of 50 cm² (reference C1PA45544) is used.

Sterilising Filtration

Sartorius Minisart filters with a surface area of 5 cm² and with porosity of 0.45 μm and 0.2 μm (references 16537 and 16532) are used.

Tests

Multicolumn Affinity Chromatography

The tests were carried out on 4 columns, connected sequentially in series during the gel adsorption and washing phases, with a load of 26 g of IgG/1 of gel, contact time of 5 minutes, binding pH between 7.3 and 7.8, elution being carried out with 0.1 M glycine solution, pH 3.

The gel was regenerated after each chromatography.

Regeneration consisted in passing 2 CV of 2 M sodium chloride solution.

The chromatography was monitored by recording OD at 280 nm and by calculating IgG yield (nephelometry assay).

The working flow rate was 2.3 ml/min, which corresponds to a contact time of 2 min during adsorption.

The multicolumn affinity chromatography steps proceeded as summarised in table 4 below.

TABLE 4
Multicolumn affinity chromatography steps

| Step | Solution | Volume | Remarks |
|---|---|---|---|
| Equilibration | Equilibration | At least 2 CV | Verification of pH |
| | buffer | | (7.4 ± 0.2) |
| Adsorption | Cryosuper- | 3 ml to 22 ml | Collection of the |
| | natant | depending on | fraction not |
| | | the test | adsorbed by |
| | | | monitoring OD at |
| | | | 280 nm |
| Washing | Equilibration | 2 to 4 CV | Until return to the |
| | buffer | | baseline |
| Elution | 0.1M Glycine, | 2 CV | Collection until |
| | pH 3.0 | | return to the |
| | | | baseline. |
| Regeneration | 2M NaCl | 2 CV | / |
| Re-equilibration | Equilibration | At least 2 CV | Verification of pH |
| of the column | buffer | | (7.4 ± 0.2) |
| Storage | 20% Ethanol | At least 2 CV | Not carried out if |
| | | | the column is |
| | | | immediately re-used |

CV: column volumes

S/D Treatment and TMAE Chromatography

S/D treatment is carried out for about 30 minutes in order to inactivate enveloped viruses. The product is then adjusted for pH and conductivity before injection on the TMAE gel.

The non-adsorbed fraction of the TMAE gel is then collected (fraction removed); after returning to the baseline and washing the column, the gel eluate is collected for the following phase.

Ultrafiltration

The ultrafiltration step makes it possible to dialyse and concentrate the eluate of the TMAE column at an intermediate concentration of 80-120 g/l.

Formulation

The product was formulated by adding formulation buffer (mannitol, glycine, polysorbate 80). The final concentration of the product is adjusted to 50 g/l.

The product obtained is sterile filtered on a sequence of 0.45 and 0.22 μm filters. It is then sampled and stored in the liquid state at a temperature of 4.0-7.0° C.

Results

Analyses were carried out on the final product for comparison with 50 g/l immunoglobulin IgG (reference product) obtained according to a method having as first purification steps fractionation with ethanol.
Assay of total IgGs and subclasses by nephelometry (Table 6).
Assay of IgAs, IgMs and IgEs by ELISA (Enzyme-Linked Immunosorbent Assay) (Table 7).

TABLE 5
Distribution of IgG subclasses

| | Total IgGs | IgG1 | IgG2 | IgG3 | IgG4 |
|---|---|---|---|---|---|
| Test | (%) | (%) | (%) | (%) | (%) |
| Final product | 100.0 | 53.4 | 33.4 | 1.2 | 1.7 |
| (IgSelect load: 26 g/l) | | | | | |
| Reference product | 100.0 | 59.7 | 36.8 | 2.4 | 1.9 |

TABLE 6
Analysis of contaminating protein content

| | Ig G | Ig A | Ig M | Ig E |
|---|---|---|---|---|
| Test | (g/l) | (mg/l) | (mg/l) | (IU/ml) |
| Final product | 57.6 | <13 | <8 | 1.5 |
| (IgSelect load: 26 g/l) | | | | |
| Reference product | 46.1 | 8.5 | <8 | <0.8 |

It is noted that these conditions for purifying IgGs directly from cryosupernatant yield a product equivalent in quality to the reference product. Moreover, as indicated in Table 6 above, conservation of all IgG subclasses is observed, with a distribution approaching that of plasma.

More interestingly, multicolumn affinity chromatography using IgSelect gel has a step yield superior to 90%, and 7.4 g of IgG collected per litre of cryosupernatant. Complete IgG depletion in the non-adsorbed fraction from the affinity gel is noted.

TABLE 7
Multicolumn IgSelect affinity yield

| | Ig G | Volume | Ig G | Step yield (%)/g of |
|---|---|---|---|---|
| IgSelect test load 26 g/l | (g/l) | (l) | (mg) | IgG per litre of plasma |
| Cryosupernatant | 8.0 | 880 | 6500 | 100/8.0 |
| Multicolumn IgSelect | 4.8 | 1245 | 6010 | 92.6/7.4 |
| affinity eluate | | | | |

In conclusion, purification of IgGs directly from plasma cryosupernatant by multicolumn chromatography is absolutely possible on an industrial scale, in particular for processing volumes of several thousand litres of plasma per day.

For example, to process an industrial batch of 4500 litres of human plasma, the following may be used: 4 columns of 50 l and a gel height of 12 cm, with a cryosupernatant load correspondent to 27 g of IgG per litre of gel, a linear adsorption flow rate between 100 and 300 cm/h, and a higher linear flow rate for the gel washing and elution steps between 200 and 600 cm/hour.

Configurations composed of 3 columns of 70 litres of gel or of 5 columns of 40 litres of gel are possible, the number of cycles to be carried out to process the totality of the batch being determined by the volume of raw material used.

Such an implementation, even on an industrial scale, makes it possible to obtain an immunoglobulin concentrate meeting regulatory requirements and having all qualities required for pharmaceutical use (purity, biological safety, IgG subclass distribution, etc.). In particular, preservation of the four IgG subclasses of the plasma is observed. In order to eliminate the solvent/detergent mixture, the IgAs and the IgEs, TMAE chromatography may then be envisaged.

Moreover, it is possible to provide for a step of washing the gel with 80% polysorbate, either simultaneously with the pre-elution with NaCl, or as a mixture in the urea/acetic acid fraction.

### Example 2

### Capture of Immunoglobulins by Multicolumn Affinity Chromatography on Plasma

Materials & Methods

Bags of human plasma having made it possible to form a pool of plasma of about 301, comprising between 8 and 10 g/l of IgG, were thawed in a 37° C. water bath without the product exceeding an internal temperature of 25° C. in order to extract the immunoglobulins via a first multicolumn chromatography.

TABLE 8
Distribution of IgG subclasses in the plasma pools

| Raw | | Total IgGs | IgG1 | IgG2 | IgG3 | IgG4 |
|---|---|---|---|---|---|---|
| material | Test No. | (%) | (%) | (%) | (%) | (%) |
| Plasma pool 1 | 1647-120 | 100.0 | 58.0 | 33.6 | 3.3 | 5.2 |
| Plasma pool 2 | 1647-121B | 100.0 | 57.4 | 32.5 | 3.4 | 6.7 |

Buffer Solutions

The composition of the buffer solutions used in the various steps of the affinity chromatography method is summarized in Table 2 below.

TABLE 9
Buffer solutions for the affinity chromatography

| Phases | Composition | Target values |
|---|---|---|
| Equilibration and washing | 10.0 mM sodium citrate, | pH = 7.4 |
| | 100 mM NaCl | |
| Pre-elution | 10 mM sodium citrate, | pH 7.4 |
| | 2.0M NaCl | |
| Elution | Acetic acid | QS pH = 3.0 |
| Eluate adjustment | 1M sodium hydroxide | QS pH 4.8 |

Gel for Chromatography

For the affinity chromatography, a CaptureSelect FcXL affinity gel from the company Life Technologies (Ref. 19432801L, Batch No. 200814-03) is used. The affinity ligand used is a ligand from the company BAC (BioAffinityCompany), which specifically binds the CH3 domain of the 4 human IgG subclasses. This ligand is a 14 kD recombinant protein.

Columns

For the multicolumn affinity chromatography, 4 radial columns from the company Proxcys, (MD 122 MK III—gel volume: 250 ml pr column for a total affinity gel volume of 1.01 of gel; gel height: 12 cm; inlet diameter/outlet diameter ratio: 2/1) were used in combination with a BioSc M pilot automated system (Novasep).

Tests

Multicolumn Affinity Chromatography

The tests were carried out on 4 columns, controlled sequentially by the automated system with a plasma load corresponding to 21 g of IgG/l of gel, a contact time of 3 to 4 minutes, at a non-modified plasma adsorption pH of between 7.1 and 7.8, elution being carried out with an acetic acid solution at pH 3.0. The gel was regenerated after each chromatography.

The chromatography was monitored by recording OD at 280 nm and by calculating the IgG yield on the pool of eluates (nephelometry assay). The multicolumn affinity chromatography steps proceeded as summarized in Table 10 below.

TABLE 10
Multicolumn affinity chromatography steps

| Step | Solution | Remarks |
|---|---|---|
| Equilibration | Equilibration buffer | (7.4 ± 0.2) |
| Adsorption | Plasma filtered 0.2 μm | Collection of the fraction |
| | | not adsorbed |
| Pre-elution | 10 mM sodium citrate, | |
| | 2M NaCl | |
| Elution | Acetic acid pH 3.0 | Collection until return to |
| | | the baseline |

In order to limit the risks of aggregation of the IgGs at excessively acidic pH, the eluates of the affinity columns obtained were adjusted to pH 4.8 using a 1 M sodium hydroxide solution.

Diafiltration Concentration of the Multicolumn FcXL Affinity Chromatography Eluate

The eluate was diafiltered in water and concentrated on a membrane having a cut-off threshold of 30 kDa in order to obtain a concentration of about 70 g/l.

Results

The analyses were carried out on the ultrafiltration-concentrated multicolumn chromatography eluate.
Assaying of total IgGs and IgG subclasses by nephelometry (Table 11).
Assaying of IgAs, IgMs and IgEs by ELISA (Enzyme-Linked Immunosorbent Assay) (Table 12).

TABLE 11
IgG subclass distribution performed after eluate
ultrafiltration and concentration

| | Total IgGs | | | | |
|---|---|---|---|---|---|
| Test | (%) | IgG1 (%) | IgG2 (%) | IgG3 (%) | IgG4 (%) |
| FcXL eluate: | 100.0 | 58.3 | 32.9 | 3.0 | 5.7 |
| plasma 1 | | | | | |
| FcXL eluate: | 100.0 | 58.5 | 34.0 | 2.2 | 5.3 |
| plasma 2 | | | | | |

TABLE 12
Analysis of the content of contaminant proteins performed
after IgG ultrafiltration and concentration

| | IgG | IgA | IgM | IgE |
|---|---|---|---|---|
| Test | (g/1) | (g/1) | (g/1) | (IU/ml) |
| FcXL eluate: plasma 1 | 76.6 | 1.00 | 0.75 | 33.0 |
| FcXL eluate: plasma 2 | 66.8 | 0.80 | 0.50 | 12.0 |

It is noted that these conditions for purifying IgGs directly from plasma give a product of very good quality. Furthermore, as indicated in Table 11 above, the preservation of all of the IgG subclasses is observed, with a distribution approaching that of plasma.

More advantageously, the multicolumn affinity chromatography using the FeXL gel has a step yield superior to 90%, that is to say superior to 7.5 g of IgG collected per litre of plasma processed. Complete IgG depletion is noted in the non-adsorbed fraction from the affinity gel. In addition, albumin and fibrinogen are collected entirely in the non-adsorbed fraction from the gel.

TABLE 13
Multicolumn FcXL affinity yield (state
of eluate adjusted to pH 4.8)

| | IgG | Volume | IgG | Step yield (%)/g |
|---|---|---|---|---|
| Test | (g/l) | (l) | (g) | IgG per litre of plasma |
| Plasma 1 | 4.4 | 60.7 | 264.0 | 92.1/7.5 |
| Plasma 2 | 3.2 | 70.4 | 225.3 | 97.9/7.9 |

In conclusion, the purification of IgGs directly from the plasma by multicolumn chromatography can be entirely envisaged on an industrial scale, in particular for treating volumes of several thousand litres of plasma per day.

TABLE 14
Comparison of IgG capture capacity with the FcXl affinity
gel in conventional chromatography and multicolumn
chromatography (equivalent chromatography buffers).

| Chromatography type | Capacity in g IgG per litre of gel |
|---|---|
| Conventional | 15 |
| Multicolumn | 21 |

Such an implementation, even on an industrial scale, makes it possible to extremely efficiently capture the immunoglobulins present in the plasma. It also makes it possible to obtain, in a single step, an eluate of great purity which retains the qualities expected of a pool of therapeutic immunoglobulins. In particular, preservation of the four IgG subclasses of the plasma and good elimination of IgAs, IgMs and IgEs are observed. In order to eliminate the residual traces of IgA, IgM and IgE, ion-exchange chromatography of TMAE type (Merck) can then be envisaged.

### Example 3

### Capture of Albumin by Multicolumn Ion-Exchange Chromatography, Hydrophobic Interaction Chromatography or a Combination of Chemical Ligands (Multi-Modal)

Materials and Methods

IgG-depleted plasma, collected at the conclusion of the method for purifying IgGs according to Example 2, is used, the albumin being not at all bound to the affinity gel used.

The characteristics of the plasma fraction are:

Human albumin at 16.15 g/l, pH=6.5, conductivity of 12 mS/cm, in 0.01 M citrate buffer.

The composition of the buffer solutions used in the various steps of the chromatography method is summarized in Table 15 below.

TABLE 15
Buffer solutions for mixed-mode multicolumn chromatography.

| Phase | Buffer/product |
|---|---|
| Pre-equilibration | 0.1M citrate, 0.1M NaCl, |
| | adjusted to pH = 6.5 |
| Equilibration | 0.01M citrate, 0.1M NaCl, |
| | adjusted to pH = 6.5 |
| Elution | 0.01M citrate and 0.1M NaCl, |
| | pH = 3.9 |

Mixed-Mode Chromatography Gels

For the albumin chromatography, a salt-tolerant mixed-mode gel of HEA Hypercel type is used.

Columns

5 radial columns in series, each of 10 ml, were used in combination with a BioSc Lab automated system (Novasep). Six cycles each of 164 minutes are carried out, i.e. about 17 hours of continuous operation.

The multicolumn chromatography conditions applied in this example made it possible to confirm virtually complete capture of the albumin, very little albumin remaining in the non-adsorbed fraction. The table below summarizes the yields in each fraction collected and the minimum purity achieved in the purified fraction of interest.

TABLE 16
Albumin yield and estimation of the
purity by SDS-PAGE electrophoresis

| Non-adsorbed | Eluate | Regeneration | Purity in |
|---|---|---|---|
| fraction yield | yield | yield | the eluate |
| 3.4% | 87.7% | 8.9% | 80% |

A gain in capacity of at least 70% is observed during the multicolumn chromatography test compared with a conventional test wherein the loading is stopped when 10% of leakage is reached at the column outlet. Table 17 summarizes the gain calculated for the 2 conditions.

TABLE 17
Comparison of the capture capacity of the HEA gel
in conventional chromatography and multicolumn
chromatography (equivalent chromatography buffers).

| | Capacity in g albumin |
|---|---|
| Chromatography type | per litre of gel |
| Conventional | 30 |
| Multicolumn | 52 |

The results from Table 17 show that only about 3.4% of the albumin was not adsorbed. An electrophoretic purity of about 80% is observed.

After the elution at pH=3.9, an 88% yield was obtained. This test shows that at least 96% of the blood plasma albumin was captured by chromatography according to a multicolumn chromatography mode on the HEA-HyperCel support and at least 88% of this albumin was collected on elution, which corresponds to a capture/elution balance of at least 80%. The multicolumn chromatography on this gel made is possible to change the capacity of the gel from 30 g/l in conventional chromatography to 52 g/l, i.e. a reduction in the total gel volume required by batch of 70%.

Since human plasma is composed of about 40 g/l of albumin, at least 33 g/l can be purified according to this example.

### Example 4

### Capture of Fibrinogen by Multicolumn Affinity Chromatography

Materials & Methods

Plasma Fraction

IgG- and albumin-depleted plasma, collected at the conclusion of the method for purifying albumin (passage over the two multicolumn chromatographies for 14 hours) according to Example 3, is used after having been frozen and stored at −80° C. and then thawed on the day of the fibrinogen capture by chromatography.

Characteristics of the plasma fraction: antigenic fibrinogen: 0.32 g/l.

Buffer Solutions

The composition of the buffer solutions used in the various steps of the chromatography method is summarized in Table 18 below.

TABLE 18
Buffer solutions for the affinity chromatography.

| Phases | Composition | Target values |
|---|---|---|
| Column equilibration | 10 mM trisodium citrate, | pH adjusted |
| and return to the | 100 mM sodium chloride | to 7.4 |
| base line | | |
| Pre-elution | 2.0M sodium chloride | pH adjusted |
| | | to 7.0 |
| Elution | 20 mM Trometamol (Tris HCl), | pH adjusted |
| | 1.5M magnesium chloride, | to 7.0 |
| | 20% v/v propylene glycol, | |
| | 200 mM arginine | |

Affinity Chromatography Gels

For the chromatography, a CaptureSelect Fibrinogen affinity gel (Life Technologies ref. 191291050, batch 171013-01) is used, the fibrinogen capture characteristics of which are summarized in Table 19 below.

TABLE 19
Capacity of the CaptureSelect Fibrinogen gel

| | |
|---|---|
| Fibrinogen load | 10 g/l of gel |

Columns

A 5 ml axial column (reference Tricorn 5/100 (GE Healthcare), column volume: 1.6 ml; column height: 8 cm) was used.

Affinity Purification

The thawed immunoglobulin- and albumin-depleted plasma is injected without modification onto the equilibrated Fibrinogen CaptureSelect affinity column. A load of about 10 g/l was applied. The fibrinogen content of the eluate was assayed by the ELISA method.

Results

At the end of the method, the fibrinogen yield is 70%. The product obtained has an antigenic fibrinogen concentration of 1.3 mg/ml.

This test clearly demonstrates that it is possible to capture the three abundant proteins of plasma by successively using only chromatography steps (apart from viral inactivation and filtration steps).

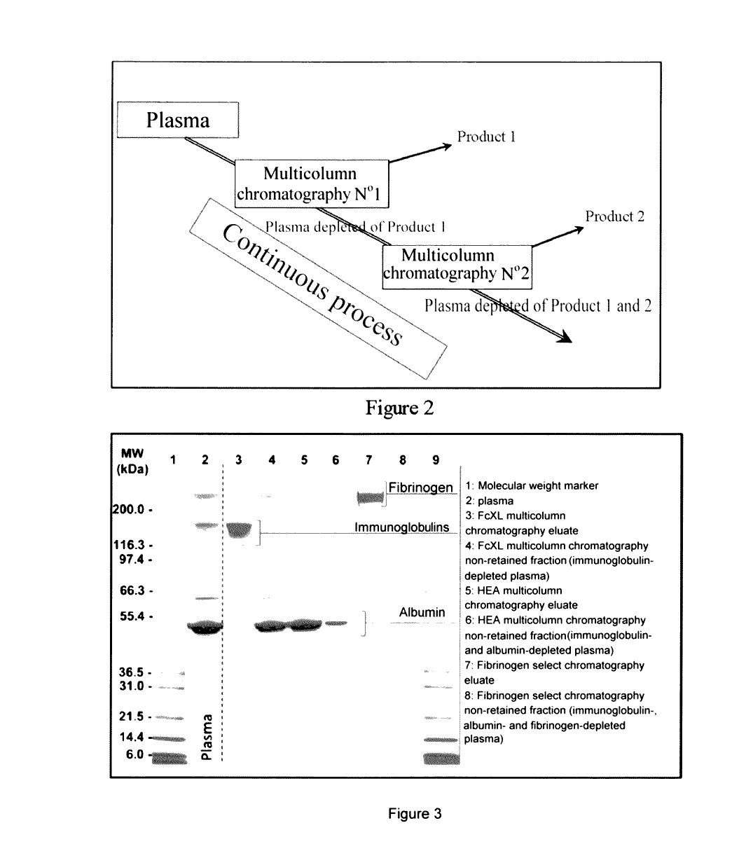

In this example, two continuous multicolumn chromatographies and a conventional chromatography were carried out one after the other, with the non-adsorbed fractions being successively reprocessed. This sequence of chromatographies (FIG. 2) over more than 10 hours is not detrimental to the quality of the molecules captured and quite particularly the molecule having been purified last, which in the present case is fibrinogen. The latter very predominantly retained its integrity throughout the purification method, as shown by the SD S-PAGE under non-reducing conditions and Coomassie blue staining of the various successive chromatographies of capture of the immunoglobulins, followed by capture of the albumin and, finally, followed by capture of the fibrinogen (FIG. 3).

In this FIG. 3, the starting plasma and then the eluates and the non-retained fractions show the successive depletion of the plasma with respect to the molecules captured and the enrichment of the eluates with captured molecules.

Well 1 corresponds to the molecular weight markers and well 2 corresponds to the unpurified normal plasma.

Wells 3 and 4 correspond to the fractions obtained in Example 2.

Wells 5 and 6 correspond to the fractions obtained in Example 3.

Wells 7 and 8 correspond to the fractions obtained in Example 4.

Fibrinogen having a molecular weight of 340 kDa predominantly retains its molecular integrity in the non-retained fraction of the multicolumn immunoglobulin capture chromatography and then in the non-retained fraction of the multicolumn albumin capture chromatography. An absence of fibrinogen is observed in the non-retained fraction of the fibrinogen capture affinity chromatography, said fibrinogen being completely captured and found with a predominantly intact structure in the eluate of this chromatography.

1. Method for preparing a purified plasma protein concentrate for therapeutic use from blood plasma, comprising a purification step wherein blood plasma or a plasma fraction is subjected to multicolumn chromatography.

2. Method according to claim 1, wherein the plasma fraction is selected from cryoprecipitated plasma supernatant, resuspended plasma cryoprecipitate, fractions I to V obtained by ethanol fractionation, supernatant and precipitate obtained after precipitation with caprylic acid and/or caprylate, eluates or fractions not retained by chromatographies, filtrates.

3. Method according to claim 1, wherein the purified plasma protein concentrate for therapeutic use contains a plasma protein selected from immunoglobulins, albumin, coagulation factors such as fibrinogen (factor I), factor VII, factor VIII, factor IX, factor XI, factor XIII, von Willebrand factor, prothrombin complex or PPSB (factors II, VII, IX, X), activated prothrombin complex, biological adhesives, and protease inhibitors such as alpha-1 antitrypsin, C1-esterase inhibitor or antithrombin, alone or in mixture, alpha/macroglobulin, antichymotrypsin, antitrypsin, Apo A, Apo B, Apo C, Apo D, Apo E, Apo F, Apo G, beta XIIa, C-reactive protein, C7, C1r, C1s, C2 C3, C4, C4bP, C5, C6, C1q, C8, C9, carboxypeptidase N, ceruloplasmin, factor B, factor D, factor H, fibronectin, haptoglobin, haemopexin, heparin cofactor II, histidine rich GP, kininase II, kininogen HPM, lysozyme, PAI 2, PAI I, PCI, plasmin, plasmin inhibitor, plasminogen, prealbumin, prekallikrein, properdin, protease nexin INH, protein C, protein S, protein Z, serum amyloid protein (SAP), TFPI, thiol-proteinase, thrombomodulin, tissue factor (TF), TPA, transcobalamin II, transcortin, transferrin, vitronectin.

4. Method according to claim 1, wherein the purified plasma protein concentrate for therapeutic use is an immunoglobulin concentrate.

5. Method according to claim 1, wherein the purified plasma protein concentrate for therapeutic use is an albumin and/or fibrinogen concentrate.

6. Method according to claim 1, wherein the purified plasma protein concentrate for therapeutic use is a fibrinogen and/or albumin and immunoglobulin concentrate.

7. Method according to claim 1, wherein the multicolumn chromatography is multicolumn affinity chromatography.

8. Method according to claim 1, wherein plasma or cryoprecipitated plasma supernatant is directly subjected to a purification step of human immunoglobulins consisting of multicolumn affinity chromatography.

9. Method according to claim 8, wherein the affinity ligand of the multicolumn affinity chromatography is selected from ligands having affinity for IgG1, IgG2, IgG3 and IgG4.

10. Method according to claim 7, wherein the multicolumn affinity chromatography uses an affinity ligand selected from antibodies, antibody fragments, antibody derivatives, chemical ligands such as peptides, mimetic peptides, peptoids, nanofitins, and oligonucleotide ligands such as aptamers.

11. Method according to claim 7, wherein the multicolumn affinity chromatography uses an affinity ligand selected from affinity ligands resistant to conditions of sanitisation and/or intensive re-use compatible with industrial use, in particular from peptides, peptoids, nanofitins and aptamers.

12. Method according to claim 1, wherein the multicolumn chromatography is multicolumn ion-exchange chromatography, such as multicolumn anion- or cation-exchange chromatography, or multicolumn hydrophobic interaction chromatography, or multicolumn mixed-mode chromatography or multicolumn size-exclusion chromatography.

13. Method according to claim 12, wherein the multicolumn anion-exchange chromatography is implemented on crosslinked polysaccharide gel or vinyl or acrylic polymer gel, grafted with DEAE or TMAE or QAE groups.

14. Method according to claim 13, wherein the multicolumn anion-exchange and/or mixed-mode chromatography is carried out on a highly salt tolerant matrix in order to capture the plasma protein in a medium having high salinity such as the plasma or fraction not retained by a chromatography.

15. Method according to claim 1, wherein plasma or cryoprecipitated plasma supernatant is directly subjected to a step of purification of human immunoglobulins consisting of multicolumn anion-exchange chromatography, and optionally wherein the fraction containing said human immunoglobulins is subjected to a subsequent purification step by precipitation with caprylic acid and the supernatant containing the immunoglobulins is collected.

16. Method according to claim 1, wherein plasma or cryoprecipitated plasma supernatant is directly subjected to a step of purification of albumin and/or fibrinogen consisting in multicolumn affinity chromatography or multicolumn anion-exchange chromatography, and optionally wherein the fraction containing albumin and/or fibrinogen is subjected to a subsequent purification step by precipitation with caprylic acid and the fraction containing albumin and/or fibrinogen is collected.

17. Method according to claim 1, wherein the multicolumn chromatography comprises from 3 to 8 columns in series, preferentially from 3 to 5 columns, and/or wherein the multicolumn chromatography columns are radial columns.

18. Method according to claim 1, comprising at least one of the following additional steps:
a delipidation and/or filtration step;
a precipitation step with caprylic acid;
a viral inactivation or elimination step, optionally including solvent-detergent treatment and/or nanofiltration;
an ion-exchange chromatography step, and in particular an anion- or cation-exchange chromatography step, the anion-exchange chromatography step being preferentially implemented on crosslinked polysaccharide gel or vinyl polymer gel, grafted with DEAE or TMAE or QAE groups;
an anti-A and anti-B antibody elimination step, in particular by affinity chromatography such as multicolumn affinity chromatography;
a concentration step by ultrafiltration;
a formulation step.

19. Method according to claim 1, further comprising a formulation step comprising the addition of one or more pharmaceutically acceptable stabilisers to the purified plasma protein concentrate for therapeutic use, and optionally a step of freezing or freeze-drying of the pharmaceutical preparation obtained at the conclusion of the formulation step.

20. Method according to claim 1, successively comprising:
(a) a first step of inactivation by solvent/detergent of the plasma;
(b) a step of purification of immunoglobulins from the inactivated plasma obtained in step (a) by multicolumn affinity chromatography; then
(c) optionally a step of anti-A and anti-B antibody elimination, in particular by affinity chromatography, from the immunoglobulin concentrate resulting from step (b); then
(d) optionally a second viral elimination step by nanofiltration of the immunoglobulin concentrate resulting from step (b) or (c); and
(e) a step of addition of one or more pharmaceutically acceptable stabilisers to the immunoglobulin concentrate resulting from step (b), (c) or (d).

21. Method according to claim 1, successively comprising:
(a′) a step of purification of immunoglobulins from cryoprecipitated plasma supernatant by multicolumn affinity chromatography; then
(b′) a first step of inactivation by solvent/detergent of the immunoglobulin concentrate obtained in step (a′); then
(c′) optionally a step of anti-A and anti-B antibody elimination, in particular by affinity chromatography, from the immunoglobulin concentrate resulting from step (b′); then
(d′) optionally a second viral elimination step by nanofiltration of the immunoglobulin concentrate resulting from step (b′) or (c′); and
(e′) a step of addition of one or more pharmaceutically acceptable stabilisers to the immunoglobulin concentrate resulting from step (b′), (c′) or (d′).

22. Method according to claim 1, successively comprising:
(A) a step of ethanol and/or caprylic acid fractionation of cryoprecipitated plasma supernatant; then
(B) optionally a first step of viral inactivation or elimination by solvent/detergent of the solution obtained in step (A); then
(C) a step of purification of immunoglobulins by multicolumn anion-exchange chromatography of the solution obtained in step (A) or (B); then
(D) optionally a step of anti-A and anti-B antibody elimination, in particular by affinity chromatography, from the immunoglobulin concentrate obtained at the conclusion of step (C); then
(E) optionally a second viral elimination step by nanofiltration of the immunoglobulin concentrate obtained at the conclusion of step (C) or (D); and
(F) a step of addition of one or more pharmaceutically acceptable stabilisers to the immunoglobulin concentrate resulting from step (C), (D) or (E).

23. Method according to claim 1, comprising steps consisting in subjecting blood plasma or cryoprecipitated plasma supernatant to:
multicolumn affinity chromatography wherein at least one affinity ligand specifically binds immunoglobulins, at the conclusion of which the fraction containing immunoglobulins is collected; and/or
multicolumn ion-exchange chromatography or multicolumn chromatography via hydrophobic interactions or a combination of chemical ligands (multi-modal), at the conclusion of which the fraction containing fibrinogen and/or albumin is collected,
and optionally a subsequent purification step by precipitation with caprylic acid of the fraction containing albumin and/or fibrinogen,
and/or optionally at least one of the following subsequent steps:
a viral inactivation or viral elimination step,
an ion-exchange chromatography step,
an anti-A and anti-B antibody elimination step, in particular by affinity chromatography,
a concentration step by ultrafiltration,
a formulation step.

24. Method according to claim 1, wherein the cryoprecipitated plasma supernatant was obtained beforehand by precipitation of blood plasma at a temperature between −10° C. and −40° C., then gentle thawing at a temperature between 0° C. and +1° C., followed by centrifugation of the thawed plasma in order to separate the cryoprecipitate and the supernatant.

25. Method according to claim 1, wherein plasma or cryoprecipitated plasma supernatant is directly subjected to a first multicolumn chromatography in order to capture a first plasma protein, then wherein the non-retained fraction from the first multicolumn chromatography is subjected to a second multicolumn chromatography in order to capture a second plasma protein.

26. Method according to claim 1, wherein the non-retained fraction from the second multicolumn chromatography is subjected to a third chromatography, preferentially a multicolumn chromatography, or to a precipitation step in order to purify a third plasma protein.

27. Method according to claim 1, wherein the retained fraction from the multicolumn affinity or ion-exchange chromatography is the plasma protein of interest, preferentially fibrinogen and/or albumin, said plasma protein of interest then being optionally eluted.

28. Method according to claim 1, wherein the retained fraction from the multicolumn affinity or ion-exchange chromatography contains the contaminants, the non-retained fraction comprising the protein of interest, preferentially albumin and/or fibrinogen.

29. Method for fractionating blood plasma, comprising steps consisting in directly subjecting blood plasma or cryoprecipitated plasma supernatant or a plasma fraction to:
multicolumn affinity chromatography wherein at least one affinity ligand is a ligand specifically binding immunoglobulins, at the conclusion of which the fraction containing the immunoglobulins is collected; and
multicolumn ion-exchange and/or mixed-mode chromatography, optionally highly salt tolerant, at the conclusion of which the fraction containing albumin and/or fibrinogen is collected; and optionally
a step of purification of said fraction not retained by the preceding multicolumn chromatography containing fibrinogen and/or albumin by precipitation and/or chromatography.

30. Method for fractionating blood plasma, comprising steps consisting in directly subjecting blood plasma or cryoprecipitated plasma supernatant or a plasma fraction to:
multicolumn affinity chromatography wherein at least one affinity ligand is a ligand specifically binding immunoglobulins, at the conclusion of which the fraction containing immunoglobulins is collected; and/or
multicolumn affinity chromatography wherein at least one affinity ligand is a ligand specifically binding fibrinogen, at the conclusion of which the fraction containing fibrinogen is collected; and/or
multicolumn affinity chromatography wherein at least one affinity ligand is a ligand specifically binding albumin, at the conclusion of which the fraction containing albumin is collected.

31. Method of fractionation according to claim 26, wherein the multicolumn affinity chromatography binding immunoglobulins is carried out upstream of the multicolumn chromatography or chromatographies binding albumin and/or fibrinogen.

32. Immunoglobulin concentrate having an immunoglobulin distribution profile similar or identical to the immunoglobulin distribution profile in plasma.

33. Immunoglobulin concentrate according to claim 32, wherein said concentrate is an immunoglobulin G concentrate having 50 to 70% IgG1, 25 to 35% IgG2, 2 to 8% IgG3 and 1 to 8% IgG4.

34. Immunoglobulin concentrate having an antigenic repertoire similar or identical to the antigenic repertoire of plasma.

35. Immunoglobulin concentrate according to claim 34, characterised in that the antigenic repertoire of said concentrate is similar and/or superior to the antigenic repertoire of concentrates of the prior art.
